(19) United States
(12) Patent Application Publication
(10) Pub. No.: US 2026/0209788 A1
(43) Pub. Date: Jul. 23, 2026

(54) LETTUCE PLANT HAVING IMPROVED RESISTANT TO DOWNY MILDEW

(71) Applicant: Enza Zaden Beheer B.V., Enkhuizen (NL)

(72) Inventors: Bas Ter Riet, Medemblik (NL); Olivia Julia Pruzina Fraser, Bristol (GB); Saskia Mulder, Grootebroek (NL); Ilja Roobeek, Dirkshorn (NL); Mathieu André Pel, Utrecht (NL)

(21) Appl. No.: 19/140,826

(22) PCT Filed: Dec. 19, 2022
(86) PCT No.: PCT/EP2022/086713

§ 371 Date: Jun. 18, 2025

(51) Int. Cl. C12N 15/82; C07K 14/415

(52) U.S. Cl. CPC C12N 15/8282; C07K 14/415

(57) ABSTRACT

The present invention relates to a lettuce plant that is resistant to downy mildew, more specifically to a lettuce plant that comprises one or more resistance genes that confers broad spectrum resistance to oomycetes in lettuce, more specifically B. lactucae. Furthermore the present invention relates to said one or more resistance genes and a method for obtaining a lettuce plant that is resistant to downy mildew, wherein the method comprises the step of identifying said one or more genes.

The present invention relates to a lettuce plant that is resistant to downy mildew, more specifically to a lettuce plant that comprises a combination of two specific resistance genes that confers broad spectrum resistance to downy mildew in lettuce, more specifically Bremia lactucae (B. lactucae). Furthermore the present invention relates to said resistance genes and a method for obtaining a lettuce plant that is resistant to downy mildew, wherein the method comprises the step of identifying said resistance genes.

Downy mildew refers to several types of oomycete microbes that are parasites of plants. Downy mildew can originate from various species, but mainly of Peronospora, Plasmopara and B. lactucae. Downy mildew is a problem in many food crops, in for example in lettuce caused by B. lactucae, affecting the production of this crop worldwide. Plants that are being affected include food crops such as brassicas (e.g. cabbage), potatoes, grape, spinach, lettuce, onion, tomato, cucumber plants. Downy mildew infection show symptoms of discoloured areas on upper leaf surfaces in combination with white, grey or purple mould located on the other side of the leaf surface below. Disease is spread from plant to plant by airborne spores.

Lettuce, mostly known as Lactuca sativa, but also including Lactuca species such as L. serriola, L. saligna or L. virosa, is a very important crop worldwide. Some of the most popular varieties available are Iceberg, Romaine, Butterhead, Batavia and Oakleaf. There are many plant pathogens that affect L. sativa, and some of the diseases caused by these pathogens are downy mildew, sclerotinia rot, powdery mildew, fusarium wilt of which the most important disease is lettuce downy mildew, which is caused by the B. lactucae, an oomycete pathogen that belong to Peronosporaceae.

For some vegetable crops, such as lettuce, cultivars with resistance to downy mildew are available. However, the pathogen under pressure will mutate to break down the disease resistance and new disease resistance in crops is needed to control infection. Especially in lettuce the occurrence of resistant downy mildew is particularly complex as there are many different races, and new resistant downy mildew species emerging all the time.

In lettuce, infection of B. lactucae result in yellow to pale green lesions that eventually become necrotic due to secondary pathogens leading to major crop losses. Fungicides can be used to control B. lactucae, but eventually B. lactucae becomes immune to these chemicals, because over time the pathogen also acquires resistance to fungicides. Furthermore, there are multiple lettuce varieties available that are resistant to B. lactucae but resistance is quickly overcome because new B. lactucae races develop rapidly. Therefore, it is of the utmost importance to find other methods to control B. lactucae infection. Most preferably is to identify a resistance gene that gives broad resistance against B. lactucae and to provide for lettuce plants that are resistant to downy mildew. Therefore, identification of resistance genes is a promising alternative.

Considering the above, there is a need in the art for to provide plants that are resistant to downy mildew and wherein plants have a broad-spectrum resistance against this pathogen. Furthermore, it is an object of present invention to provide a method to obtain such downy mildew resistant plants.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a lettuce plant having improved resistance to downy mildew, wherein said plant comprises a RCB1 gene and a RCB2 gene, wherein said RCB1 gene encodes for a RCB1 protein having at least 93% sequence identity with SEQ ID No. 2, preferably at least 95%, more preferably at least 98%, even more preferably at least 99%, most preferably 100% with SEQ ID No. 2, and wherein said RCB2 gene encodes for a RCB2 protein having at least 99% sequence identity with SEQ ID No. 4, preferably 100% with SEQ ID No. 4. The RCB1 gene and/or RCB2 gene are dominant resistance genes, and may be homozygous or heterozygous present in a downy mildew resistant lettuce plant, preferably homozygous present.

The majority of disease resistance genes in plants encode nucleotide-binding site leucine-rich repeat proteins, also known as NBS-LRR proteins (encoded by R genes). These proteins are characterized by nucleotide-binding site (NBS) and leucine-rich repeat (LRR) domains as well as variable amino- and carboxy-terminal domains and are involved in the detection of diverse pathogens, including bacteria, viruses, fungi, nematodes, insects and oomycetes. There are two major subfamilies of plant NBS-LRR proteins defined by the Toll/interleukin-1 receptor (TIR) or the coiled-coil (CC) motifs in the amino-terminal domain and are both involved in pathogen recognition, probably via the LRR domain. A leucine-rich repeat (LRR) is a protein structural domain composed of repeating 20 to 30 amino acid stretches that forms an horseshoe fold. The domain is rich in the hydrophobic amino acid leucine. The region between the helices and sheets is the protein's hydrophobic core and is tightly sterically packed with leucine residues. The RCB1 gene is not a classical NBS-LRR gene, it is an RLK-LRR protein. The LRR motif of RCB1 contains leucines in a beta-sheet and the domain forms a horseshoe structure in total. Furthermore, the RCB1 gene contains a (pseudo) kinase domain, in which the RCB1 gene differs from other cases of R genes where multiple LRR regions and the NBS domain are present together with TIR or CC domain. It is thought that those domains determine effector recognition and therefore disease susceptibility/resistance. The presence of the RCB1 resistance gene will decrease the chances of the pathogen overcoming the resistance, as often seen with the R genes. Even so, combined with R genes, disease resistance (e.g. against downy mildew) may even be further improved.

The RCB1 gene is a not a classical R gene since it contains a (pseudo) kinase domain together with a LRR domain, whereas the RCB2 is a gene with only a LRR domain. The RCB2 gene is a classical R gene as described above, whereas the RCB1 gene is a kinase/pseudokinase with an LRR domain. RCB1's ability to bind ATP and potential phosphorylation activity may be key to the downstream immune response following B. lactucae detection. It is likely that RCB1 and RCB2 interact in protein form either as a result of B. lactucae detection or in the absence of it. The detection of B. lactucae by either RCB1 or RCB2 could lead to a change in conformation, enabling potential kinase action by RCB1 on other downstream proteins. The B. lactucae resistant plant of present invention therefore combines the action of an RLK-LRR gene containing a (pseudo) kinase domain and a LRR domain and a R gene with LRR domain, providing full spectrum B. lactucae resistance in lettuce plants. The combination of the RCB1 and RCB2 genes of present invention provides resistance to B. lactucae races B11 to B135, more preferably B1:1 to B1:37.

To demonstrate that the RCB1 and RCB2 genes are related to B. lactucae resistance, this putative resistance gene has been silenced by tobacco rattle virus (TRV)-based virus-induced gene silencing (VIGS) to induce susceptibility to B. lactucae infection in resistant lettuce plants containing these resistance gene. With VIGS it was demonstrated that both the RCB1 and RCB2 genes were associated with the observed downy mildew resistance, VIGS gene silencing was used to create B. lactucae-susceptibility in resistant Lactuca species. Resistant lettuce plants were transient transformed independent with RCB1 and RCB2 silencing construct and made susceptible to B. lactucae infection, thus by removing/reducing expression of these gene via virus induced gene silencing.

According to another preferred embodiment, the present invention relates to the Lettuce plant, wherein the RCB1 gene encodes for cDNA sequence having at least 95% sequence identity with SEQ ID No. 1, and wherein RCB2 gene encodes for cDNA sequence having at least 99% sequence identity with SEQ ID No. 3.

According to yet another preferred embodiment, the present invention relates to the lettuce plant, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

According to a preferred embodiment, the present invention relates to the lettuce plant, wherein the lettuce plant is a Butterhead, Iceberg, Romaine, Batavia or Oakleaf lettuce, preferably a Butterhead lettuce.

According to another preferred embodiment, the present invention relates to the lettuce plant, wherein downy mildew is caused by B. lactucae, wherein the downy mildew is caused by one or more of B. lactucae selected from the group of race B1:1 to B1:37, preferably resistant to all of B1:16 to B1:37, more preferably resistant to all of B1:1 to B1:37. B. lactucae races have been characterized and classified according to the SEXTET code by IBEB (International Bremia Evaluation Board).

According to yet another preferred embodiment, the present invention relates to the lettuce plant, wherein the RCB1 gene, and RCB2 gene are obtainable from deposit number NCIMB 44030. Seeds of this line are deposited at NCIMB Ltd, Aberdeen, Scotland on 9 Sep. 2022 under the number NCIMB 44030.

According to another preferred embodiment, the present invention relates to the lettuce plant, wherein the plant further comprises an MACPF resistance gene on Chr9, wherein the MACPF resistance gene comprises a coding sequence having at least 95% sequence identity with SEQ ID No. 18, preferably 100% with SEQ ID No. 18. The MACPF resistance gene can be stacked with the RCB1 and RCB2 genes on chromosome 9 in the lettuce plant of present invention, further providing strong resistance to B. lactucae. The MACPF resistance gene in lettuce has been described previously in WO2020/035145.

The present invention, according to a second aspect, relates to seed or plant parts of a lettuce plant of present invention. The seed fruits or plant parts comprise the RCB1 and RCB2 genes as described above.

The present invention, according to a further aspect, relates to a resistance gene RCB1 that confers resistance to B. lactucae in lettuce plants, wherein the gene comprises a coding sequence having at least 95% sequence identity with SEQ ID No. 1, preferably at least 96%, more preferably at least 98%, most preferably at least 99%, most preferably 100% (i.e. the coding sequence of SEQ ID No. 1). The RCB1 gene is a dominant gene. SEQ ID No.1 represents the coding nucleotide sequence of RCB1 gene and encodes encodes for a RCB1 protein having at least 93% sequence identity with SEQ ID No. 2, more preferably at least 96%, even more preferably at least 99%, preferably 100% with SEQ ID No. 2. Lettuce plants that express this protein show complete resistance to downy mildew.

The present invention, according to a further aspect, relates to a resistance gene RCB2 that confers resistance to B. lactucae in lettuce plants, wherein the gene comprises a coding sequence having at least 99% sequence identity with SEQ ID No. 3, most preferably 100% (i.e. the coding sequence of SEQ ID No. 3). The RCB2 gene is a dominant gene. SEQ ID No.3 represents the coding nucleotide sequence of RCB2 gene and encodes the gene encodes for a RCB2 protein having at least 99% sequence identity with SEQ ID No. 4, preferably 100% with SEQ ID No. 4. Lettuce plants that express this protein show complete resistance to downy mildew.

The present invention, according to a further aspect, relates to a combination of resistance gene RCB1 and resistance gene RCB2 that confer resistance to B. lactucae in lettuce plants.

According to another preferred embodiment, the present invention relates to the resistance gene RCB1, RCB2, or a combination of RCB1 and RCB2 as disclosed herein, wherein resistance to B. lactucae in lettuce comprises resistance to races B1:1 to B1:37, preferably to B1:16 to B1:37.

The present invention, according to a further aspect, relates to a method for obtaining a lettuce plant that is resistant to downy mildew, wherein the method comprises the steps of,
a) crossing a lettuce plant comprised of a resistance gene RCB1 and/or a resistance gene RCB2 as disclosed herein at least one time with a lettuce plant that does not comprise one or both of said genes, providing a lettuce plant that comprises both of said RCB1 and RCB2 genes,
b) optionally, selfing the plant obtained in step a) for at least one time,
c) selecting the plants that are resistant to downy mildew.

In the method of present invention the lettuce plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

According to another preferred embodiment, the present invention relates to the method, wherein the lettuce plant that comprises both said RCB1 and RCB2 genes is furthermore crossed with a lettuce plant comprising an MACPF resistance gene comprises a coding sequence having at least 95% sequence identity with SEQ ID No. 18, preferably 100% with SEQ ID No. 18.

The present invention, according to a further aspect, relates to a method for identifying (i) a downy mildew resistant lettuce plant of present invention or (ii) a seed of said plant, the method comprises the step of establishing, in the genome of a plant or seed the presence of a RCB1 and RCB2 resistance gene encoding, respectively, a RCB1 and RCB2 protein as described herein.

According to another preferred embodiment, the present invention relates to the method, wherein the step of establishing, comprises establishing the presence of SEQ ID No. 1 and SEQ ID No. 3 in said plant, and/or,
wherein the step of establishing, comprises establishing the presence of a resistance locus flanked by flanking markers of SEQ ID No. 5 in combination with SEQ ID No. 6.

According to a preferred embodiment, the present invention relates to the methods, wherein the RCB1 and/or RCB2 gene are provided in the resistant lettuce plant of present invention by gene editing techniques, preferably by mutagenesis and/or CRISPR/Cas. A lettuce plant comprised of the RCB1 and/or RCB2 gene gives a high downy mildew resistance phenotype. A plant having this resistant phenotype can be obtained via use of gene editing and/or mutation techniques, such as EMS mutagenesis or CRISPR/Cas in concert with cloning techniques on the RCB1 and/or RCB2 gene to generate disease resistant crops. Alternatively, a RCB1 and/or RCB2 gene can be brought into the plant by means of transgenic techniques or by introgression.

The present invention, according to a further aspect, relates to the use of a gene construct plasmid for introducing one or more resistance gene(s) into the genome of a plant or plant cell, wherein the gene construct is comprised of a resistance gene RCB1 and/or a resistance gene RCB2 operably linked to expression providing sequences in said plant. The resistance gene(s) of present invention may be transferred (e.g. by transformation or transfection) into plants, such as lettuce plants, using a plasmid of vector or linear gene construct that comprises the RCB1 and/or RCB2 resistance gene(s) of present invention. The Resistance gene(s) RCB1 and/or RCB2, after being transferred into the lettuce plant would provide resistance to B. lactucae, i.e. resistance to B. lactucae race B1:1 to B1:37, preferably B1:16 to B1:37.

The present invention will be further detailed in the following examples and figures wherein:

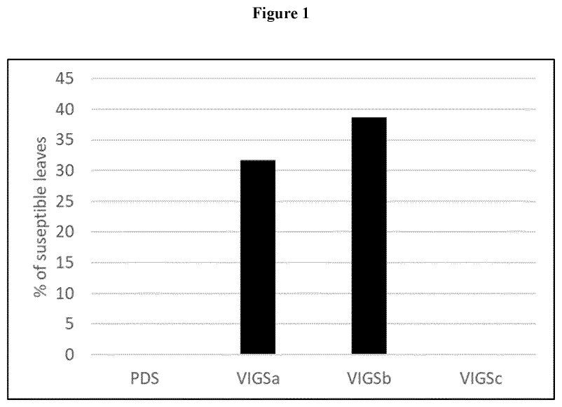

FIG. 1: shows the % of leaves of Lettuce (Y-axis) that are susceptible to B. lactucae after VIGS silencing of RCB1 (by VIGSa), RCB2 (by VIGSb), PDS (by VIGS_PDS) and NC (by VIGSc) (X-axis). The RCB1, RCB2, PDS and NC(=Lsa17111) gene has been silenced in these plants using VIGS gene silencing and subsequently infected with B. lactucae (B122). On the x-axis from left to right: sample leaves of plants in which the PDS gene (control), RCB1, RCB2 and part of the resistance locus (another control) is silenced using silencing constructs, PDS, VIGSa, b and c, respectively. In the samples with a resistant phenotype, there is no B. lactucae present. In the samples with susceptible phenotypes, B. lactucae is present. As expected with transient gene silencing, VIGS gene silencing does not result in fully 100% silencing of the genes in all plants. However, the leaves from plants wherein the resistance gene RCB1 or RCB2 has been silenced by VIGS silencing, showed a high number of susceptible leaves when infected with B. lactucae as compared to plants where said RCB genes were not silenced, i.e. plant is both control groups were resistant to B. lactucae infection. No susceptible leaves were observed when RCB1 or RCB2 expression was not affected by VIGS.

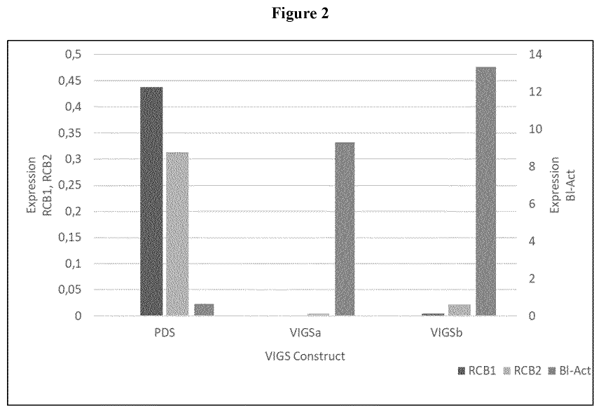

FIG. 2: shows B. lactucae actin (Bl-Act), RCB1 and RCB2 gene expression levels in Lettuce after VIGS gene silencing as described in FIG. 1 infected with B. lactucae (B122), determined by qPCR. The B. lactucae expression levels in the leaves of plants that showed to be resistant or susceptible to downy mildew after gene silencing, were collected and RNA was isolated to determine the expression levels of B. lactucae by qPCR. The transcription levels of B. lactucae lactuca was determined by the transcripts of a B. lactucae house keeping gene (actin) in relation to the lettuce house keeping gene TUA-3. Leaves of the plants that were resistant to B. lactucae showed to have a high RCB1 and RCB2 gene expression and low transcriptional levels of the B. lactucae house keeping gene. In case RCB1 or RCB2 gene expression levels were VIGS silenced in lettuce infected with B. lactucae (B1:22), expression levels of B. lactucae actin increased dramatically. Leaves of the plant that were susceptible to B. lactucae, showed high transcriptional levels of the B. lactucae house keeping gene actin, indicating the susceptibility corresponds with low RCB1 or RCB2 gene expression due to VIGS silencing.

### EXAMPLES

### Gene Mapping of Resistance Gene in Lettuce

Gene mapping experiments were done to identify a resistance gene that is involved in full spectrum B. lactucae (B. lactucae) resistance in wild lettuce varieties, such as L. serriola. The resistance gene was mapped on chromosome 9 on the lettuce genome (https://lgr.genomecenter.ucdavis.edu). Furthermore, we were able to reduce the size of the introgression fragment comprising the RCB1 and RCB2 genes providing full spectrum B. lactucae resistance in lettuce, to a RCB resistance locus of about 60 kb. The RCB resistance locus comprising the RCB1 and RCB2 genes is identified by flanking Marker 1 (position 218655172 on lettuce genome) and Marker 2 (position 218716325) of Table 1.

TABLE 1
Marker sequences

| Marker | Sequence |
|---|---|
| Marker 1 | GGAATGTTACACCAAGAACCTGTAC |
| (SEQ ID No. 5) | ACATATCTGAAAATCCATTGTGTTT |
| | AAATTTTAAACTTTTTTTTGCGTTG |
| | TCATATTTTTTTGAACATGAGCACA |
| | TTCTG |
| Marker 2 | CTATCGCATGCCCCTGCTGCATCAA |
| (SEQ ID No. 6) | TATTGCTCCCGTACCTATGATCGAA |
| | GCGTTACAGTAGACTACCAAGTCAT |
| | TTACTCCCTTTCGTAGGGTAAGAAT |

After fine mapping in a population of about 12,000 plants there were three putative resistance genes present in the identified resistance locus. Using VIGS analysis (see below), two resistance genes could be identified: RCB1 and RCB2.

### RCB1 and RCB2 Resistance Gene Silencing Experiment Using Virus Induced Gene Silencing (VIGS)

To demonstrate that the RCB1 and RCB2 resistance genes provide B. lactucae resistance, the RCB1 and RCB2 resistance gene were silenced by tobacco rattle virus (TRV)-based virus-induced gene silencing (VIGS) to induce susceptibility to B. lactucae infection in L. sativa lines containing both resistance genes. Tobacco rattle virus (TRV)-derived VIGS vectors have been abundantly described to study gene function in Arabidopsis thaliana, Nicotiana benthamiana, Solanum esculentum and other plants (see for example Huang C, Qian Y, Li Z, Zhou X.: Virus-induced gene silencing and its application in plant functional genomics. Sci China Life Sci. 2012; 55 (2): 99-108). With VIGS it was demonstrated that both the RCB1 and RCB2 resistance gene are associated with downy mildew resistance, since VIGS induced gene silencing was used to create B. lactucae susceptibility in resistant Lactuca accessions comprising the RCB1 and RCB2 gene. Resistant lettuce plants were transiently transformed with a silencing construct specific against either the resistance RCB1 or the RCB2 gene which will result in the silencing of the respective resistance gene. It was found that both RCB1 and RCB2 are required for and were involved in the resistant phenotype observed in Lettuce.

Briefly, lettuce plants containing the RCB1 and RCB2 resistance genes were silenced for the RCB1 or RCB2 resistance gene by VIGS using different silencing construct to identify if this RCB1 and/or RCB2 resistance gene was/were indeed responsible for the observed resistance.

Several VIGS-constructs were designed, one (VIGSa) that results in specific silencing of the RCB1 gene, VIGSb that specifically silenced the RCB2 gene, and VIGSc that targets a gene in the 60 kb resistance locus (but not the RCB1 or RCB2 gene). VIGS off target effects or silencing cross-reactivity of the VIGS constructs is prevented such that all three constructs are very different in sequence, i.e. targeting specifically RCB1, RCB2 or different sequence on the resistance locus, respectively. Furthermore, independent of resistance gene silencing the PDS gene is silenced as well that serves as positive control to indicate if VIGS is working and to determine the efficiency. The PDS gene is involved in carotenoid biosynthesis and is the first step in lycopene biosynthesis. This step is catalyzed by the enzyme phytoene desaturase (PDS). When silencing of the PDS gene is achieved, this results in bleached leaves. Experiments showed bleached leaves indicating that the VIGS silencing was achieved and performed correctly (data not shown). All plants that were VIGS inoculated were harvested and put in a tray and sprayed with B. lactucae to test the effect of the gene silencing on disease resistance.

The VIGS constructs were cloned in the K₂O vector (See Table 2). The constructs were transformed and transiently expressed into a lettuce plant of present invention that is resistant to B. lactucae, using co-cultivation with agrobacterium (GV3101) to study the resistance gene function in relation to B. lactucae resistance. The % of susceptible B. lactucae leaves was observed in both groups and both silencing constructs. With the leaves of VIGS-experiments independent disease tests (see below) were performed to observe that when RCB1 or RCB2 gene was silenced, plants became susceptible to B. lactucae.

Results (FIG. 1) indicate that when RCB1 or RCB2 was silenced by VIGS with the VIGSa or VIGSb construct the plants became susceptible after B. lactucae infection (B122) confirming that both of the RCB1 and 2 resistance genes are linked to the plant resistance against B. lactucae. Thus independent if RCB1 or RCB2 was silenced, plants became susceptible. When plants were silenced with PDS or VIGSc (both control groups), the plants remain resistant to B. lactucae. Therefore, it can be concluded that both RCB1 and RCB2 are involved providing B. lactucae disease resistance in lettuce plants.

TABLE 2
VIGS constructs

| VIGS-constructs | Sequence |
|---|---|
| VIGSa | CACCCAAAATTTCAAAGAAAGCTTG |
| (SEQ ID No. 7) | CTCATAGGGACAGGTGGATTCGGGA |
| | AGTTGTACAAAGGCACAATCAAGAA |
| | TGGAACAACAACCAGTATTGTGGCT |
| | GTTAAGCAGTGGAACCACGCTTTTA |
| | ATCAAGGCGCCCAAGAGTTTCAGGC |
| | TGAAGTTCAATTAGTTTCTATGTTG |
| | CGACACCCTAACATAGTGTGTTTGA |
| | TTGGATATTGTACTGATGACAAAGA |
| | GATGATCCTTGTTTACGAATATATG |
| | TCCAATGGAAGCCTTGAAGAGCACC |
| | TACACAAGCACAATACTCAATTATC |
| | TTG |
| VIGSb | GATGATGTTAAGGAAGATCTGAGCC |
| (SEQ ID No. 8) | TAGATAAGATTGGAGTTCCTATAAG |
| | CGAGAATGGTAGCAAAATAGTATTT |
| | ACAACTAGGTTACGACATGTTTGCT |
| | CCTCAATTGCAACAAGACAAACCAA |
| | TGTTGCAACATTAAACAGAAAGGAA |
| | GCATTGGATATGTTCAAAAGTGTAT |
| | TGGATCGACCACATCTTGCAGAAAA |
| | CTCCAACATTAAACGTCTAATGCCT |
| | CTTATAGTCAATTGGTGTGGTTATC |
| | ATCCACTCATGATAAAGGTAGCAGC |
| | AGGTGTGTTTAGAGTGAAGGAGACC |
| VIGSc | CATGGGGCAATCAGTTACTTTGGGG |
| (SEQ ID NO. 9) | GTACCAAGAATCCATGGACACTGAG |
| | CTTCAACAAATTTTTAACCTAGCTG |
| | TCGATAATAGTATTAACCTCTTTGA |
| | AACCGCTGATTCTTATGGTACTGGT |
| | AAACTAAATGGGCAGAGCGAGAAGC |
| | TTATTGGAAGGTTCATAGGTGAATT |
| | TAAAGGACAGACACAAGTTCGTCAT |
| | AACATTGTGATCGCCACAAAGCTTG |
| | TTGCATATCCATGGCGCCTGACACC |
| | AAACCAATTTGTGAATGCGTGCAAG |
| | TATAGTATACACGTCTCTTCAGTCT |
| | TG |

### Disease Test and Biotest for Downy Mildew in Lettuce

Leaves of resistant plants transiently transformed with the above described VIGS constructs, were put in trays with moistened paperboard and infected with B. lactucae. Infected seedlings are suspended in 20 mL water, filtered by cheesecloth and the flow-through is collected in a spray flask. The trays are spray-inoculated with the B. lactucae suspension. The trays are covered with a glass plate and stored in a climate chamber at 15° C. (12 hours of light). A black, opaque foil is placed over the trays for one day to improve growth of B. lactucae. After one day, the foil is removed. Experiments were performed in triple, and eight to ten days after infection leaves are phenotypically scored by eye on the presence of B. lactucae, i.e. being susceptible or resistant.

A lettuce plant (L. sativa) of present invention comprising the RCB1 and RCB2 resistance genes was used to test B. lactucae diagnostic. Seeds of this line are deposited at NCIMB Ltd, Aberdeen, Scotland on 9 Sep. 2022 under the number NCIMB 44030. Disease resistance tests show that plants comprising the RCB1 and RCB2 resistance genes are resistant to B. lactucae races from B16 to B137EU, no disease symptoms were observed in the plants invented with the B. lactucae races. Furthermore, further disease resistance test show that plants comprised of the RCB1 and RCB2 resistance gene are also resistant to US B. lactucae race B1:9. Previous disease resistance test have shown that the RCB1 and RCB2 resistance gene also provides resistance to B1:1 to B1:15, therefore the combined RCB1 and RCB2 resistance genes provides full spectrum resistance to B1:16 to B1:37, and even more preferably to B1:1 to BL: 37EU.

Determine B. lactucae Expression in Lettuce Comprising the RCB1 and RCB2 Genes

Gene expression experiments were conducted in lettuce tissues obtained from the VIGS experiment as outlined above, to determine RCB1 and RCB2 expression. To obtain more insight in the response of lettuce to infection with B. lactucae, leaves of resistant and susceptible plants used in the VIGS experiment as described above were harvested. cDNA was synthesized from RNA that had been isolated from infected leaves. The expression of RCB1 and RCB2 gene was assessed in lettuce by conducting qPCR. Expression of B. lactucae actin and expression of RCB1 and RCB2 were analyzed by qPCR using the primers as set out in Table 3.

TABLE 3

| | | SEQ |
|---|---|---|
| Primer | | ID |
| name | Sequence | No. |
| RCB1-F | TGGTCGCAAAGGCTTCAGAT | 10 |
| RCB1-R | CCTCTTGCCCACTACCACTG | 11 |
| RCB2-F | CAATTGGGCAGCAGACGAAC | 12 |
| RCB2-R | ATGCGGCTAGGCGAATCAAT | 13 |
| B. lactucae | GCGAGAAATTGTGCGTGATA | 14 |
| actin-F | | |
| B. lactucae | ACTCGGCTGCAGTCTTCATT | 15 |
| actin-R | | |
| LsTUA-3-F | CTTCTTAGTGTTCAATGCTGTTGG | 16 |
| LsTUA-3-R | GAAGGGTAGATAGTGAAACCGAGC | 17 |

FIG. 2 shows the results of a qPCR of the housekeeping gene B. lactucae actin (Bl-Act) and RCB1 and RCB2 resistance gene expression levels in Lettuce after VIGS gene silencing. The transcription levels of B. lactucae lactuca was determined by the transcripts of a B. lactucae housekeeping gene (actin) in relation to the house keeping gene TUA-3 of lettuce. Values on the y-axis are relative CT values (the fold increase is calculated as 2\hat{ }-(Ct B. lactucae actin-Ct TUA3A). On the x-axis from left to right: sample leaf of a plant of present invention (i.e. comprising the RCB1 and RCB2 genes) in which PDS is silenced, sample leaves of said plants in which the RCB1 gene is silenced using silencing construct VIGSa, sample leaves of said plants in which the RCB2 gene is silenced using silencing construct VIGSb. In the samples with the observed and expected resistant phenotype, i.e. the PDS control, there is no or almost no B. lactucae present. In the samples with susceptible phenotypes, high transcription levels of the housekeeping gene B. lactucae actin were measured, whereas low levels of the silenced target genes were measures.

Leaves of the plants that were resistant to B. lactucae showed to have a high RCB1 and RCB2 gene expression and low transcriptional levels of the B. lactucae housekeeping gene. In case RCB1 or RCB2 gene expression levels were VIGS silenced in lettuce infected with B. lactucae (B122), expression levels of B. lactucae actin increased dramatically. Leaves of the plant that were susceptible to B. lactucae, showed high transcriptional levels of the B. lactucae housekeeping gene actin, indicating the susceptibility corresponds with low RCB1 or RCB2 gene expression due to VIGS silencing.

1. A lettuce plant having improved resistance to downy mildew, wherein said plant comprises a RCB1 gene and a RCB2 gene, wherein said RCB1 gene encodes for a RCB1 protein having at least 93% sequence identity with SEQ ID No. 2, and wherein said RCB2 gene encodes for a RCB2 protein having at least 99% sequence identity with SEQ ID No. 4.

2. The lettuce plant according to claim 1, wherein the RCB1 gene encodes for cDNA sequence having at least 95% sequence identity with SEQ ID No. 1, and wherein RCB2 gene encodes for cDNA sequence having at least 99% sequence identity with SEQ ID No. 3.

3. The lettuce plant according to claim 1, wherein the lettuce plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

4. The lettuce plant according to any one of the claim 1, wherein the lettuce plant is a Butterhead, Iceberg, Romaine, Batavia or Oakleaf lettuce, preferably a Butterhead lettuce.

5. The lettuce plant according to claim 1, wherein downy mildew is caused by Bremia lactucae (B. lactucae), and/or wherein the lettuce plant is resistant to downy mildew caused by one or more of B. lactucae selected from the group of races B1:1 to B1:37, resistant to all of B1:16 to B1:37, or resistant to all of B1:1 to B1:37.

6. (canceled)

7. The lettuce plant according to any of the claim 1 wherein the RCB1 gene is at least heterozygous present in the genome of the plant, or homozygous, and/or
wherein the RCB2 gene is at least heterozygous present in the genome of the plant, or homozygous.

8. (canceled)

9. The lettuce plant according to claim 1, wherein the RCB1 gene, and RCB2 gene are obtainable from deposit number NCIMB 44030.

10. The lettuce plant according to claim 1, wherein the plant further comprises an MACPF resistance gene on Chr9, wherein the MACPF resistance gene comprises a coding sequence having at least 95% sequence identity with SEQ ID No. 18, or 100% with SEQ ID No. 18.

11. A seed or plant part of a lettuce plant according to claim 1.

12. A resistance gene RCB1 that confers resistance to B. lactucae in lettuce plants, wherein the gene comprises a coding sequence having at least 95% sequence identity with SEQ ID No. 1, or 100% with SEQ ID No. 1.

13. The resistance gene RCB1 according to claim 12, wherein the gene encodes for a RCB1 protein having at least 93% sequence identity with SEQ ID No. 2, or 100% with SEQ ID No. 2.

14. A resistance gene RCB2 that confers resistance to B. lactucae in lettuce plants, wherein the gene comprises a coding sequence having at least 99% sequence identity with SEQ ID No. 3, or 100% with SEQ ID No. 3.

15. The resistance gene RCB2 according to claim 14, wherein the gene encodes for a RCB2 protein having at least 99% sequence identity with SEQ ID No. 4, or 100% with SEQ ID No. 4.

16. A combination of resistance gene RCB1 and resistance gene RCB2,
wherein the resistance gene RCB1 comprises a coding sequence having at least 95% sequence identity with SEQ ID No. 1, or 100% with SEQ ID No. 1,
wherein the resistance gene RCB2 comprises a coding sequence having at least 99% sequence identity with SEQ ID No. 3, or 100% with SEQ ID No. 3, and
wherein the combination confers resistance to B. lactucae in the lettuce plant according to claim 1.

17. The combination of RCB1 and RCB2 according to claim 16, wherein resistance to B. lactucae in lettuce comprises resistance to races B1:1 to B1:37, and/or to B1:16 to B1:37.

18. A method for obtaining a lettuce plant that is resistant to downy mildew, wherein the method comprises the steps of,
a) crossing a lettuce plant comprised of a resistance gene RCB1 and/or a resistance gene RCB2 according to claim 16 at least one time with a lettuce plant that does not comprise one or both of said genes, providing a lettuce plant that comprises both said RCB1 and RCB2 genes,
b) optionally, selfing the plant obtained in step a) for at least one time,
c) selecting the plants that are resistant to downy mildew,
wherein the lettuce plant is selected from Lactuca sativa, Lactuca virosa, Lactuca saligna, Lactuca serriola, Lactuca aculeate, Lactuca georgica, Lactuca perennis, Lactuca tatarica, and Lactuca viminea.

19. (canceled)

20. The method according to claim 18, wherein the lettuce plant that comprises both said RCB1 and RCB2 genes is furthermore crossed with a lettuce plant comprising an MACPF resistance gene comprises a coding sequence having at least 95% sequence identity with SEQ ID No. 18, or 100% with SEQ ID No. 18.

21. A method for identifying (i) a downy mildew resistant lettuce plant according to claim 1 or (ii) a seed of said plant, the method comprises the step of establishing, in the genome of a plant or seed the presence of a RCB1 and RCB2 resistance gene encoding, respectively, a RCB1 and RCB2 protein as defined in claim 1.

22. The method according to claim 21, wherein the step of establishing, comprises establishing the presence of SEQ ID No. 1 and SEQ ID No. 3 in said plant,
and/or,
wherein the step of establishing, comprises establishing the presence of a resistance locus flanked by flanking markers of SEQ ID No. 5 in combination with SEQ ID No. 6.

23. Use of a gene construct plasmid for introducing one or more resistance gene(s) into the genome of a plant or plant cell, wherein the gene construct is comprised of a resistance gene RCB1 and/or a resistance gene RCB2 according to claim 16 operably linked to expression providing sequences in said plant.


SEQUENCE LISTING (18 sequences)

SEQ ID NO: 1 - DNA, 5334 bp, Lactuca sativa
  source 1..5334: mol_type = other DNA
atgtggagaagcgatgattccctttacagttcctcatggtcgtccaaaccgtccaatttgccacctccacctgcatcgagtaaggagaccggatcatcgtccccaaagggacaactttgccgtcaatttacgattgatgagattcgatccgccacccaaaatttcaaagaaagcttgctcatagggacaggtggattcgggaagttgtacaaaggcacaatcaagaatggaacaacaaccagtattgtggctgttaagcagtggaaccacgcttttaatcaaggcgcccaagagtttcaggctgaagttcaattagtttctatgttgcgacaccctaacatagtgtgtttgattggatattgtactgatgacaaagagatgatccttgtttacgaatatatgtccaatggaagccttgaagagcacctacacaagcacaatactcaattatcttggtcgcaaaggcttcagatatgtattgatgttgcacgtgcattacactacctccacagtggtagtgggcaagaggtcatacacggcaatcttacaagctcagatattttgttagatagccagtggggagccaaaatcacagactttggattgtctaaaataatctctttcgatgactgctctacccatgttagtactgctgtttctgccttttcaggcacgcttggggatattgatccagaatatatggcaacaggcagactaagcaagaagtctgatgtgtattcctttggagtggttttgttggaagtgttatgcaggaggcgtgtagttgatacaggtcttaaaaaggaacagcaaggactggtgagatgggctcaagactgtgtgagaagagggaaactaaagcagatcatagatccagagttgaaggggcacattttacatacgtgcttgaatgattatgtgaaaatagtggaagagtgtttgcgtgaaagtcaaaatcaacgtgttacaatggatgaggttgtttctggtcttgagtctatattggcattacaagagagaagtgatcataaatctattccccaaatgggtataaccacatctgattggaggttggataagcaattctcccatggacgaagaaactctgggggaagctactttagcaaggtcacaaggcttttgtctgccaaatccctcctgcataagggagatgaaggtaacatatccaaactgtccaatttgccacctgtatctacatcatcgtccccaaagggagaacgtttccatcaatttacaattgctcaaattcaatccgccactcaagacttcaatgaaagcttggggaaaggtggattcgggaacgtgtacaaaggcacaatcaagaatggaataacaacaactactgtggctgttaagaggtggaagtccacttcatcccaaggcatccaagagtttaagactgaagtgtatttacttcctttgttgcaacaccctaacatagtgtgtttgattggatattgtaccgatgccaaagagatgatccttgtttacgaatatatgtccaatggaagccttggagaccatttaaacaagcggagcactcctttatcttggtcacaaaggctcaaaatatgtcttgaggttgcacgtgcattagtatacctccacaatgggagtagtgggcaagaggtcatatacaacaatgtgaatagcgcagatattttgttagataaccagtgggtagcaaaaatcgcagactttggattgtctaaaacaatcccttttgataactaccctaccgatgtaattactgccgttgtaggcacagttgggtatttagatccagaatatgtggcaactggcagactaagtaagaagactgatgtgtattcctttggagtgcttctattggaagtgctatgcaggaggcgtgcaatcgatttaagtcttgttcaggaacaagtagaattggtgagatgggctcaagactgtttgagactagggaaaccagagcagatcattgatccagagttgaggggtcggatttcacttaaatgcttaaatgattatgtaagaatagtggaatgctgtttgcgtgatcgtcaagagccacgtattacaatggctcaggttctttttggtcttgagtctataatggcattacatgagagaactgatcataattcaattccccaaatgggtataactacatctgattggaggttgcagaaatattttttttcatccggagagaactcagggggaagctactttagagacggcagagagccggaaaaggattttaatgttatatggaaaccatctgaaatgactccaatgagaagtatccggttctctgatgaggattctgatcaagctagtgagccagacgatgaagtcattgttcaagagttgccatctgaagaagaaaccatgaagtccttgtatgaattgatgcagttggagtatatacaacgagctggtggattgatgaggatgattgactttttacacctcatctggttttaccctatgaaaaaagaagcggaggtcaactttgggtctcttttagacaagttttcctcatttaataagtacattagaaagttacaagactcttcaagggatgaacgctacctgatggagattcaaaaacttcttcaagacatcgaagaagatttagatgatgctgcttttgatatggtggcctatttaccagaaggcttgcaagttcctttagcaagtttttggtcaggtataccagaagtccatgcaagtgcaattgcttcagccactcaacgagctataagttgtataactgatttgaaagtcaagaagcttggaactgcaggaagtggtgctcttgaaatagccgaaatattaaaggatatgccagagttaagaagagcctttgatatggtgttgtgtgtacatgtaaaacatgatagcatcgaagagcttatgaatgacatagaagaagagatgcatttgtggaggaaaagaagtttggaaactggcagtaaaataaacgttctgatcaataagttcccaaactgcttgttattcgtagattgcagtgacacttatatcgatttgcatgaccctgagttcaatctttccaaatggtttggaacagtgcagatagtgattacaagtggatcagaaaatgcttattgcccagtggatatagagatcagagttgaggatcatcttctaccttggatattattctctacaaatgttgacctagaaacagtgggcaagtactctgaaattcaacaaatggcagcacaattaatcgaaaagtgcaatggccatttacttgccatcattttacttgcaagggctttgagaggcgtggttgatgttggtgtatgggtacttgcatttcacgaattatcctcactcgagaacccttcttcatcatcatcctcactagagaagccttcttcatcttcatcatcacagatgggtgtcacaaatgatgtcatggtgagggtattgagattcatttggtctcgcatggaatccttgtctcagaggtgcatcgtacaattcgcatcacactacataggatccaaaataaaaaagtcttctttgataaacagttggactaaagatggcctggtaaaaggtgaacaagaagcagaagatgtctttgaagatgttatacggtctttccttatagagcaagtcgggggaaattgtgttagaatgagaaatgagattcgagttattttagttacttactttgtagagtctctacctcgtggatatggactttatcctaaacaagatagttcagaaccaaacaaaatgccaaacattgaagagtcggatgcctgggaaatccatttaagtaataacataatatccgagcttccaaataaccccaactgcccccttcttgtgaatttgttcttgcacttcaatcaagatctgactgatattcctgtcacattcttcgataacatgccgagcctccaagttctagatttgtccagcacgagcataaaatgtttgccatcttccatttctaagttgacagctcttgggaaattattcataagagactgtgaccttttgatggaactaccacctgaaattggagcccttaagaatctcaaggtctttgactcagaaggaacacaacttgtatgcttaccagagcaatttgggtcgctaaccaagttggaatgcttgaagttttctttgtacaaacaaagcaaccaaagtatgcaaataattcttgcagcagttttgtcaaagctcctacggctaaaggaactaagcgtttgtgtggatttgtatggtgaatggtgggaagatgaggtcaaacttatcatcaacattttgcctaaatttctgaacttgaaatctctgacattgtatttcccaaccacagaacttctgggcatatttatggagacacagagctggcgaaaggtcccaatttaccaagatttgtcgaattttggtttcattgttggtcaccttcaacaacgtatgatatcccgtgtcccacttgaccttactaagacctttgcaaaactgccaaagtgcctgacatatacaaatggcgagggggacacacaagtgatttccagggtgctaaaacatgctaattctcttttcttagatcgccactggactctccagtccttgtctgcctttaggctggcgaagatggagaagctgaaattctgcatgttatcggaatgcaatgagatgctgcagattgtcaatggacaccatttagaatatatttatttttttgtaagaccagttcttgtatcattacaacatctctcagttcattacatgaagagtctcctttgcatctggaatgggccaattcatagtcggtgtctgtccaatctaaaaaccttggcgatgcattcatgccctgagttgaggaccatttttacgcaggggttgcttaaaagtctcacttgcttggagtgccttatcgttgaagactgcagcacaataaaaaccctagtaagtttggaatcgccccagtctacatcaactccttgtctcccaagcttaaagaagatatcacttatccacctcccagaattgttgagcatctctggtggtataagtattgccccaagattagaaagtctggtcgtttatgattgcccaaaccttgagaaactatcctacatgaaagcatttgataataataatatcaaggaaattaaaggagagaatgaatggtgggatgcattgaaatggtgcgaaccggagtggaccggtggtcggcctgattatctggctagagttttcattccgcttggaacaaatggagacattatggatgaactagctgatgctgtcaacattctccctcatttaagtgattag

SEQ ID NO: 2 - protein, 1777 aa, Lactuca sativa
MWRSDDSLYSSSWSSKPSNLPPPPASSKETGSSSPKGQLCRQFTIDEIRSATQNFKESLLIGTGGFGKLYKGTIKNGTTTSIVAVKQWNHAFNQGAQEFQAEVQLVSMLRHPNIVCLIGYCTDDKEMILVYEYMSNGSLEEHLHKHNTQLSWSQRLQICIDVARALHYLHSGSGQEVIHGNLTSSDILLDSQWGAKITDFGLSKIISFDDCSTHVSTAVSAFSGTLGDIDPEYMATGRLSKKSDVYSFGVVLLEVLCRRRVVDTGLKKEQQGLVRWAQDCVRRGKLKQIIDPELKGHILHTCLNDYVKIVEECLRESQNQRVTMDEVVSGLESILALQERSDHKSIPQMGITTSDWRLDKQFSHGRRNSGGSYFSKVTRLLSAKSLLHKGDEGNISKLSNLPPVSTSSSPKGERFHQFTIAQIQSATQDFNESLGKGGFGNVYKGTIKNGITTTTVAVKRWKSTSSQGIQEFKTEVYLLPLLQHPNIVCLIGYCTDAKEMILVYEYMSNGSLGDHLNKRSTPLSWSQRLKICLEVARALVYLHNGSSGQEVIYNNVNSADILLDNQWVAKIADFGLSKTIPFDNYPTDVITAVVGTVGYLDPEYVATGRLSKKTDVYSFGVLLLEVLCRRRAIDLSLVQEQVELVRWAQDCLRLGKPEQIIDPELRGRISLKCLNDYVRIVECCLRDRQEPRITMAQVLFGLESIMALHERTDHNSIPQMGITTSDWRLQKYFFSSGENSGGSYFRDGREPEKDFNVIWKPSEMTPMRSIRFSDEDSDQASEPDDEVIVQELPSEEETMKSLYELMQLEYIQRAGGLMRMIDFLHLIWFYPMKKEAEVNFGSLLDKFSSFNKYIRKLQDSSRDERYLMEIQKLLQDIEEDLDDAAFDMVAYLPEGLQVPLASFWSGIPEVHASAIASATQRAISCITDLKVKKLGTAGSGALEIAEILKDMPELRRAFDMVLCVHVKHDSIEELMNDIEEEMHLWRKRSLETGSKINVLINKFPNCLLFVDCSDTYIDLHDPEFNLSKWFGTVQIVITSGSENAYCPVDIEIRVEDHLLPWILFSTNVDLETVGKYSEIQQMAAQLIEKCNGHLLAIILLARALRGVVDVGVWVLAFHELSSLENPSSSSSSLEKPSSSSSSQMGVTNDVMVRVLRFIWSRMESLSQRCIVQFASHYIGSKIKKSSLINSWTKDGLVKGEQEAEDVFEDVIRSFLIEQVGGNCVRMRNEIRVILVTYFVESLPRGYGLYPKQDSSEPNKMPNIEESDAWEIHLSNNIISELPNNPNCPLLVNLFLHFNQDLTDIPVTFFDNMPSLQVLDLSSTSIKCLPSSISKLTALGKLFIRDCDLLMELPPEIGALKNLKVFDSEGTQLVCLPEQFGSLTKLECLKFSLYKQSNQSMQIILAAVLSKLLRLKELSVCVDLYGEWWEDEVKLIINILPKFLNLKSLTLYFPTTELLGIFMETQSWRKVPIYQDLSNFGFIVGHLQQRMISRVPLDLTKTFAKLPKCLTYTNGEGDTQVISRVLKHANSLFLDRHWTLQSLSAFRLAKMEKLKFCMLSECNEMLQIVNGHHLEYIYFFVRPVLVSLQHLSVHYMKSLLCIWNGPIHSRCLSNLKTLAMHSCPELRTIFTQGLLKSLTCLECLIVEDCSTIKTLVSLESPQSTSTPCLPSLKKISLIHLPELLSISGGISIAPRLESLVVYDCPNLEKLSYMKAFDNNNIKEIKGENEWWDALKWCEPEWTGGRPDYLARVFIPLGTNGDIMDELADAVNILPHLSD

SEQ ID NO: 3 - DNA, 2967 bp, Lactuca sativa
  source 1..2967: mol_type = other DNA
atggctgaagcaatagcagctggtgctggtgtggcagcagcaattatccaaggtacgacagcattatgggagccacttaagcaaacaattgcattatcaaaagatatggacgaaatatatggagctttggatgatgcaatgcaaaccctcattgcgaaaagggatgatcatgaggacatggttcaaaggcacacgacaacaatgacaccatccaatgcttacaccaactggattcacagagtaaatttggttgcaaaagaagtgaacgatttaaaagccaaatatacaaaagaaaggaaaaggtcatcatggttttctattggttcacgttctagcttcactaagaagatgaggaagactgcctcacgggtctttgctcttatggaagaagatacacatttggtagatgttttggttgacaaaaaacctgcatgtgttgtggagatgataacttcaaaaattacaaatattccaactctccaacagccattagaacaaacattgggttggttatgtgacaaaagggttagaggcattagaatccatggacttattgggagtgggaaaacaacgataatggagaatttgaataaccatgtcaaggttgctgaaatgtttgatatagtaatctgggtaacagtatcaagagaaggaagcatcgagaacagaggcatcgaccaaattcagcaagttattatccaaagattgaaactaaatatcggggttaccactaccattgacctaattgcttctagaataagagaagagttgacaaacatcaagtacttgctacttcttgatgatgttaaggaagatctgagcctagataagattggagttcctataagcgagaatggtagcaaaatagtatttacaactaggttacgacatgtttgctcctcaattgcaacgagacaaaccaatgttgcaacattaaacagaaaggaagcattggatatgttcaaaagtgtattggatcgaccacatcttgcagaaaactccaacattaaacgtctaatgcctcttatagtcaattggtgtggttatcatccactcatgataaaggtagcagcaggtgtgtttagagtgaaggagaccgaagagagttggcatgatggtttaataaacttgagaaaatggccagaaaaaggagatgataccatgcaagaaatatacaagttattggcatgtttttttgattccttgaaagttgtccaaaagaaatgcttcttttatagtgcattgtatccggaagatagtgacattcaaaaggattgcctactagacaattgggcagcagacgaacttctcgacacagttgatgatgtggaaggaacacgtgttagtggacgtgacatattggattatcttaagatgctctcattggttgaggaaaatactcaacagtgtattaggatgcacaaattgattcgcctagccgcattatataatttgtcaactaacaaagaccaagaaaatctaataaaatttggtgaagcattgcataagcccttggatgtggaatgttggaaaaacaagagatggatctcactcgttgacagtaaaatgaatgcattacccgacgaaccagattgtccaaagctttcgacgctatttttgcagaaaaatcctaatttggaagtcattccacgagcattctttaatcatatgcaaggtcttcgtgtccttgacttatatgatacgagaatttcatcattaccaagctctgttttgaacctagtcaccttgaaagttctctatctgcaaaaatgtgtggccttagttgagcttcctcatgatattggaaatcttcaaaaactcgaagtgctcgatacccgtggcagcggattagcaaacataccacctcaagttgagagattgatctgtctaaggcgtttgttggtatctttcactagctcatcaagtagctctcctattcaagaagcaactgaagacatcaaagtaatctccaagattcctaagcttaaagaattggtaattgatgttgaggaagggctagagaaacttttatatggtttgattgtcaatgttgttacgttaacaaatctttcacttcttcaattttgttctcctaacaaggtggtagatctcatcaaagctataggcggttgttggaagatcaactttcctaatgaaaaacttcttcaaaagtacatcgacaaggtagaaagctctaaactcgaaaactgccaaatttttataggttgtgatatctcatcgcctcctcacattatgaagttcatgccatatttcaagttcaatggagaagttagcaataacacaatctcacaagtacttggtaatgatgttgcaattgaattggtgaatcataatgaacttgagcacttatccaacttcactcctgcaaacatgaacaaaatccgtagttgtgttatcaaaggttgcaacaagatgaggaccatagtggatggtaacacgggaggtagcttgatattaaacaatttggaacaattacacatgatcaatttgccatttttgaagaacatctcagaaggtccaatgcaaggtctaaataacttgaaaactttgattttatgtaactgtcccatgttgacatgggtatgtacatatggtgtaattctacaacttcccgagattcgtcatctagaagtcaaagggtgctccgagatgaaagaaatattcagtgggcctgtaaatgttgtttctccggtgcttccaaatctaaagaaactaatacttgttgatatgcctcaacttacaagcatactggtaagaaactcatttgagtggccttctttggagagacttaagatccttgggtgtcccgaattgaaaagactaccttttcgaaaagatggtgctgcaaagctgaaatccatcgaaacagagaaaagttggtgggaagctttagaaaatgacaaggatgttaaagaacaatttcgacgctattgcaccttgaggtaa

SEQ ID NO: 4 - protein, 988 aa, Lactuca sativa
MAEAIAAGAGVAAAIIQGTTALWEPLKQTIALSKDMDEIYGALDDAMQTLIAKRDDHEDMVQRHTTTMTPSNAYTNWIHRVNLVAKEVNDLKAKYTKERKRSSWFSIGSRSSFTKKMRKTASRVFALMEEDTHLVDVLVDKKPACVVEMITSKITNIPTLQQPLEQTLGWLCDKRVRGIRIHGLIGSGKTTIMENLNNHVKVAEMFDIVIWVTVSREGSIENRGIDQIQQVIIQRLKLNIGVTTTIDLIASRIREELTNIKYLLLLDDVKEDLSLDKIGVPISENGSKIVFTTRLRHVCSSIATRQTNVATLNRKEALDMFKSVLDRPHLAENSNIKRLMPLIVNWCGYHPLMIKVAAGVFRVKETEESWHDGLINLRKWPEKGDDTMQEIYKLLACFFDSLKVVQKKCFFYSALYPEDSDIQKDCLLDNWAADELLDTVDDVEGTRVSGRDILDYLKMLSLVEENTQQCIRMHKLIRLAALYNLSTNKDQENLIKFGEALHKPLDVECWKNKRWISLVDSKMNALPDEPDCPKLSTLFLQKNPNLEVIPRAFFNHMQGLRVLDLYDTRISSLPSSVLNLVTLKVLYLQKCVALVELPHDIGNLQKLEVLDTRGSGLANIPPQVERLICLRRLLVSFTSSSSSSPIQEATEDIKVISKIPKLKELVIDVEEGLEKLLYGLIVNVVTLTNLSLLQFCSPNKVVDLIKAIGGCWKINFPNEKLLQKYIDKVESSKLENCQIFIGCDISSPPHIMKFMPYFKFNGEVSNNTISQVLGNDVAIELVNHNELEHLSNFTPANMNKIRSCVIKGCNKMRTIVDGNTGGSLILNNLEQLHMINLPFLKNISEGPMQGLNNLKTLILCNCPMLTWVCTYGVILQLPEIRHLEVKGCSEMKEIFSGPVNVVSPVLPNLKKLILVDMPQLTSILVRNSFEWPSLERLKILGCPELKRLPFRKDGAAKLKSIETEKSWWEALENDKDVKEQFRRYCTLR

SEQ ID NO: 5 - DNA, 105 bp, synthetic construct
  source 1..105: mol_type = other DNA
ggaatgttacaccaagaacctgtacacatatctgaaaatccattgtgtttaaattttaaacttttttttgcgttgtcatatttttttgaacatgagcacattctg

SEQ ID NO: 6 - DNA, 100 bp, synthetic construct
  source 1..100: mol_type = other DNA
ctatcgcatgcccctgctgcatcaatattgctcccgtacctatgatcgaagcgttacagtagactaccaagtcatttactccctttcgtagggtaagaat

SEQ ID NO: 7 - DNA, 303 bp, synthetic construct
  source 1..303: mol_type = other DNA
cacccaaaatttcaaagaaagcttgctcatagggacaggtggattcgggaagttgtacaaaggcacaatcaagaatggaacaacaaccagtattgtggctgttaagcagtggaaccacgcttttaatcaaggcgcccaagagtttcaggctgaagttcaattagtttctatgttgcgacaccctaacatagtgtgtttgattggatattgtactgatgacaaagagatgatccttgtttacgaatatatgtccaatggaagccttgaagagcacctacacaagcacaatactcaattatcttg

SEQ ID NO: 8 - DNA, 300 bp, synthetic construct
  source 1..300: mol_type = other DNA
gatgatgttaaggaagatctgagcctagataagattggagttcctataagcgagaatggtagcaaaatagtatttacaactaggttacgacatgtttgctcctcaattgcaacaagacaaaccaatgttgcaacattaaacagaaaggaagcattggatatgttcaaaagtgtattggatcgaccacatcttgcagaaaactccaacattaaacgtctaatgcctcttatagtcaattggtgtggttatcatccactcatgataaaggtagcagcaggtgtgtttagagtgaaggagacc

SEQ ID NO: 9 - DNA, 302 bp, synthetic construct
  source 1..302: mol_type = other DNA
catggggcaatcagttactttgggggtaccaagaatccatggacactgagcttcaacaaatttttaacctagctgtcgataatagtattaacctctttgaaaccgctgattcttatggtactggtaaactaaatgggcagagcgagaagcttattggaaggttcataggtgaatttaaaggacagacacaagttcgtcataacattgtgatcgccacaaagcttgttgcatatccatggcgcctgacaccaaaccaatttgtgaatgcgtgcaagtatagtatacacgtctcttcagtcttg

SEQ ID NO: 10 - DNA, 20 bp, synthetic construct
  source 1..20: mol_type = other DNA
tggtcgcaaaggcttcagat

SEQ ID NO: 11 - DNA, 20 bp, synthetic construct
  source 1..20: mol_type = other DNA
cctcttgcccactaccactg

SEQ ID NO: 12 - DNA, 20 bp, synthetic construct
  source 1..20: mol_type = other DNA
caattgggcagcagacgaac

SEQ ID NO: 13 - DNA, 20 bp, synthetic construct
  source 1..20: mol_type = other DNA
atgcggctaggcgaatcaat

SEQ ID NO: 14 - DNA, 20 bp, synthetic construct
  source 1..20: mol_type = other DNA
gcgagaaattgtgcgtgata

SEQ ID NO: 15 - DNA, 20 bp, synthetic construct
  source 1..20: mol_type = other DNA
actcggctgcagtcttcatt

SEQ ID NO: 16 - DNA, 24 bp, synthetic construct
  source 1..24: mol_type = other DNA
cttcttagtgttcaatgctgttgg

SEQ ID NO: 17 - DNA, 24 bp, synthetic construct
  source 1..24: mol_type = other DNA
gaagggtagatagtgaaaccgagc

SEQ ID NO: 18 - DNA, 1775 bp, Lactuca sativa
  source 1..1775: mol_type = other DNA
atggcgcttaagcttccagctgctgaagctgcaaatgttgcgattcagtcaattggatgtggttatgatatcgcgttagatctgaggcttaattatcgcatgcgtcgttatcattcagatattggttctaatccatatagaaattgccgtttgatagaaattgaggaagatgaaggtagagatattgtgcttcctggtgggcttctacttctcaatgttcccgaatccatcaaatgtcatgaaagcgagtatacacgacttcattctgatgttctttcttttccgcagatgtcggagcaattcaatcaggaattatcattatccggcaaaattccttcaggcctcttcaattatgtgtttgatttttctggaaattggaagaaagatgcatcaagtactaaaaccctagcttttgatggtgttttcatctctttgtacacggttgcattagaagaatcacacatggttctttgtgatcacgtcaagaaagctgttccatcttcatgggagcctgctttgttagcaaggtttatggaaaaatttggcactcatgtaattgtgggtgtaaaaatgggtggaaaagatgtgatatatatgaaacaacagcatgcatcatcccttgaacctgctgatgtacagaaaaagttaaaggaaatggcagacaagagatttttggactctaaacagatttcacaaaatgacacgtttgacatcacacatctaagatctgcagacttagacacctctagctcttattcttacacaaaaaatgacaaggagcatcttttaagcatttgcagaaggagaggtgggagtgatgacaggaacctaaaacataacgagtggttacaaaccgtacaatcagaaccagatgtaatcacaatgtcctttgttccaataagcttgttgctaaagggtgttatgggaagtggattcttaagtcatgccataaatctttatttacgctataaacctccatttgaagagcttcctcagtttctggacttccagctaccgacacagtgggcccctgtattcggtttgttgtctcgtggtccacaacatgagcagcgaagtgtgacatctttacagttcagcttttttgggaccaagctctacgtgaacacctatcctgtcgatgtaggtaagaggcctgtaacaggcctccgactttccctcaattgtgaaagaaacaatcagttagagatccatctccagcacctctccgccgtcccaaaattcgtccaactagaagactccgtaaccgaaatcctcaatttcgattcccacgatcataaagactacgaaaaggtccaagcgaaaaatttcgaccatgtttgcacagcctctgtcgagtctgaagacgaattcgcaatcgtaaccggggcccagttacatgtcggggatcacgggtctaaaaaagtcctcttcctacgcctccacttctcaaaactacatggttgcatcgcaaataggaatccggaatgggaagtgtctccggttcagaaactggattcttcaatttctttcatggaggtggtctacgcacgacaatttcaagtcccgcatgccccacttcacgaaccgaaatttgtgggcccgacggagacgatgcagaagctattgaagtttgtggacacaacggagatgaagcggggcccgcaagatagcccagggtattgggttgtaataggggctagacttgttgtggataagggaaagatttctcttcgagtcaagtacgctttgcctcagatattaatcgatgattagaa